(19) United States
(12) Patent Application Publication
(10) Pub. No.: US 2005/0027170 A1
(43) Pub. Date: Feb. 3, 2005

(54) Surgical retractors

(75) Inventors: Yutaka Nohara, Koshigaya-shi (JP); Kazuya Oribe, Tokyo (JP); Hiroshi Takamido, Nagoya-shi (JP)

Correspondence Address: GREENBLUM & BERNSTEIN, P.L.C., 1950 ROLAND CLARKE PLACE, RESTON, VA, 20191, US

(73) Assignee: Showa Ika Kohgyo Co., Ltd., Aichi (JP)

(21) Appl. No.: 10/878,045

(22) Filed: Jun. 29, 2004

(30) Foreign Application Priority Data
Jul. 10, 2003 (JP) P2003-272999

(51) Int. Cl. A61B 1/32

(52) U.S. Cl. 600/219

(57) ABSTRACT

The present invention provides surgical retractors that can prevent their own arms and handle portions from being raised even when a deep portion of a surgical incision needs to be held open. The surgical retractors are provided with a pair of arms which are openable and closable via a hinge portion and include a blade for spreading and holding an incision open at an extremity portion of the arms and a pair of handles for performing an open and close operation of said pair of arms, the handles being provided on said arms respectively. At the extremity portion of each arm is provided a bent arm portion that bends downward in relation to the arm. A bend line L, formed at the extremity portion of said blade provided at the extremity portion of the bent arm portion, is substantially parallel with said arm when seen from a lateral side.

### CROSS REFERENCE TO RELATED APPLICATION

This application claims benefit of priority under 35 U.S.C. § 119 to Japanese Patent Application No. 2003-272999, filed on Jul. 10, 2003, the entire contents of which are incorporated by reference herein.

### BACKGROUND OF THE INVENTION

1. Field of the Invention

The present invention relates to surgical retractors for holding open an incision made for example for a surgical operation. More particularly, the present invention pertains to surgical retractors that can prevent a handle portion of the surgical retractors from being raised higher even if a deep portion of the incision needs to be held open by a blade.

2. Description of the Related Art

Surgical retractors for ensuring an operative field by spreading and holding open a surgical incision have been disclosed, for example, in Japanese Publication of Registered Utility Model Applications 2,131,233. The surgical retractors disclosed in the publication are obtained by devising a so-called nasal speculum and allow the incision to be spread by inserting an extremity portion thereof into the incision and closing grippers. However, the surgical retractors have a drawback in that it is difficult to hold open the incision to a fixed extent because the grippers have to be kept closed by the user during the operation.

An ecarteur for holding a surgical incision to a fixed extent has been disclosed in Japanese Patent Application Laid-open H09-234,204. The ecarteur has a pair of branch portions disposed in parallel with each other, each of the base edge thereof being connected to each other by means of a connecting portion. With this connecting portion, each of extremity portions thereof is capable of moving to and away from each other elastically. In addition, the ecarteur has a positioning member, disposed between the pair of branch portions, for regulating the position of the pair of branch portions, thereby allowing the distance between the extremity portions thereof to be kept constant during operation.

Another example of conventional surgical retractors has been described in “MIZUHO” ORTHOPEDIC IMPLANTS & INSTRUMENTS (MIZUHO Co., Ltd., December 2002). As shown in FIG. 1, the surgical retractors 1 for holding open an incision that has been made, for example, over a patient's spinal cord have been comprised of a pair of arms 5A, 5B that are openably and closably connected with each other by means of a hinge portion 7, the arm 5A(5B) having at its extremity portion a blade 3 for spreading and holding the incision open. Also, the arm 5A(5B) is integrally provided with a handle 9A(9B) for performing an open/close operation of the pair of arms 5A, 5B. At the end of the handles 9A, 9B are provided respectively rings 11A, 11B for user's fingers to be inserted.

When a user inserts his or her fingers into the rings 11A, 11B and closes the handles 9A, 9B, the arms 5A, 5B pivot around the hinge 7 and the extremity portions of the arms 5A, 5B open accordingly. In order to hold the extremity portions open, the handles 9A, 9B are provided at a position adjacent to the hinge 7 with a lock mechanism 13 for locking the handles 9A, 9B against each other.

The lock mechanism 13 is comprised of a plate 15 that is formed into an arc shape around the hinge 7 and secured on one handle 9A, a ratchet finger member 17 that is pivotably provided on the other handle 9B, the end portion of the member 17 being capable of meshing with a ratchet teeth portion 15A formed on the peripheral of the plate 15, and a leaf spring 19 that is provided on the handle 9B in order to deflect the ratchet finger member 17 into engagement with the ratchet teeth portion 15A.

In case of the conventional surgical retractors 1 having such a configuration, when the blades 3 are inserted deep into an incision made on a patient body in an attempt to spread and hold open a deep portion of the incision, the arms 5A, 5B, the handles 9A, 9B, and rings 11A, 11B extrude aslant from the incision. In other words, the deeper the blades 3 are inserted into the incision, the steeper the inclination of the arms 5A, 5B, the handles 9A, 9B, and rings 11A, 11B becomes and, in a worst case, they come to stand substantially vertically from the incision.

So, there must be a problem in that the arms 5A, 5B, the handles 9A, 9B, and rings 11A, 11B tend to interfere with surgical operation.

### SUMMARY OF THE INVENTION

The present invention, which has been made in light of the aforementioned problem, provides surgical retractors provided with a pair of arms which are openable and closable via a hinge portion and include at an extremity portion thereof a blade for spreading and holding open an incision and a pair of handles for performing an open and close operation of said pair of arms, the pair of handles being provided on said pair of arms respectively, wherein there is provided at an extremity side of each of said arms a bent arm portion that bends downward in relation to the arm, and a bend line, formed at an extremity portion of said blade provided at an extremity portion of the bent arm portion, is substantially parallel with said arm when seen from a lateral side.

Also, the bent arm portion is provided at the end thereof with a blade back portion that extends substantially parallel with the arm when seen from a lateral side, and the blade back portion is provided with the blades.

According to the present invention, since blades for holding open a surgical incision when the incision needs to be held open are provided at an end of the bent arms that are formed at an end of the arms so as to bend downward and the bend line is parallel with the arm when seen from a lateral side, even when the blade is inserted deep beneath a skin in the incision, the bend line at the end of the blade can be maintained substantially parallel with the surface of the patient's body. Therefore, the arms and handles are prevented from being raised, thereby solving the aforementioned problem.

### BRIEF DESCRIPTION OF THE DRAWINGS

In the accompanying drawings:

FIG.1 is an illustration of conventional surgical retractors;

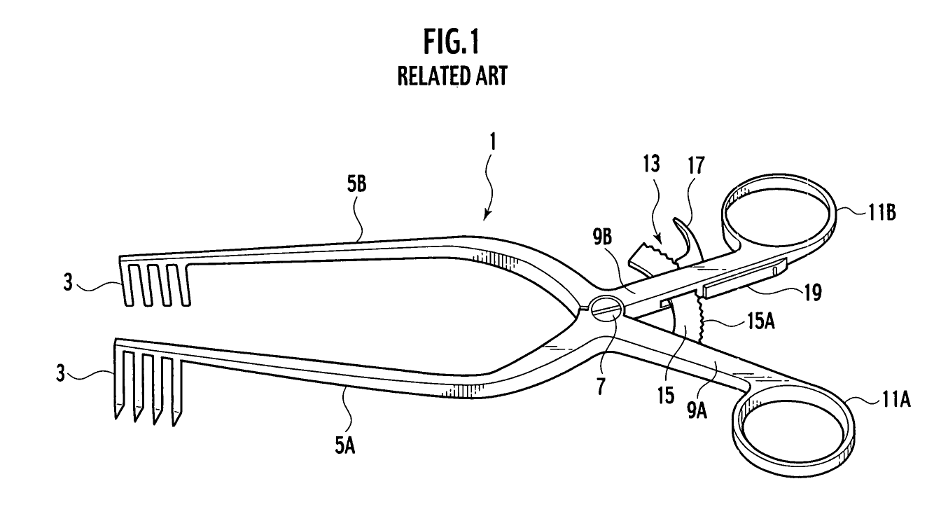

FIG.2 is a perspective illustration of surgical retractors according to the present invention;

FIG.3 is a lateral illustration of surgical retractors according to the present invention; and,

FIG.4 is an illustration of surgical retractors according to the present invention in use for a surgical incision.

### DESCRIPTION OF THE PREFERRED EMBODIMENT

Surgical retractors according to an embodiment of the present invention will be explained in detail hereinafter with reference to the accompanying drawings. In the drawings, an identical reference numeral is used to specify an identical element that demonstrates the same function as the aforementioned conventional one does, in order to avoid undue repetition in explanation.

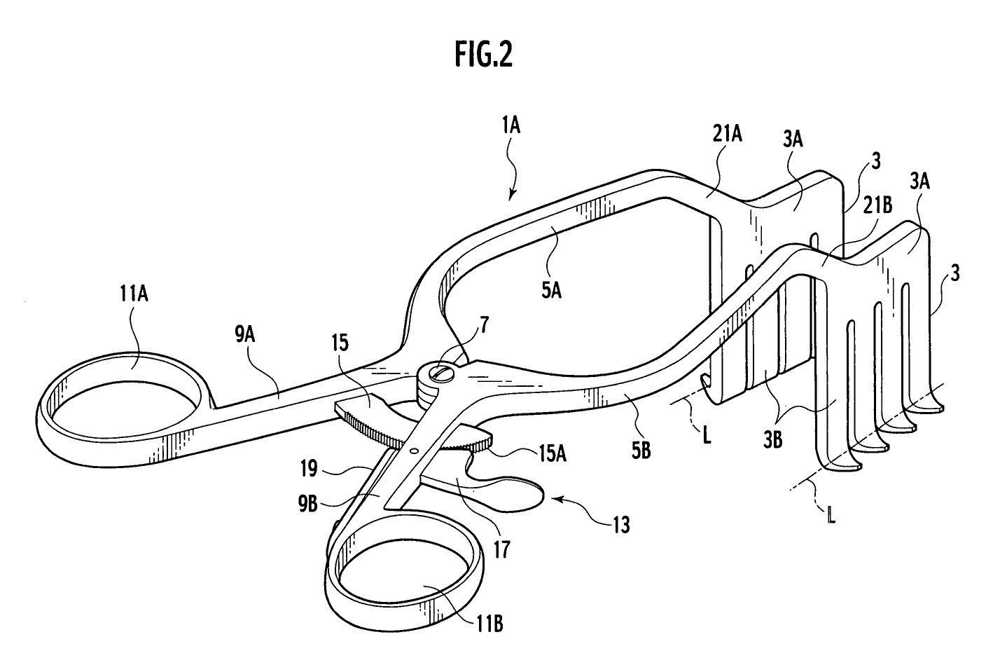

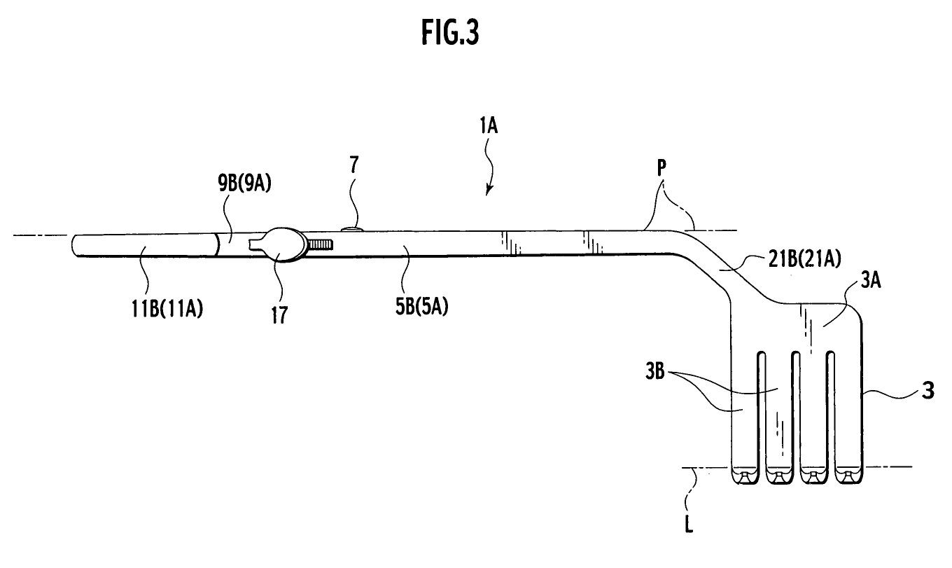

Referring to FIGS. 2 and 3, surgical retractors 1A according to an embodiment of the present invention are composed of a pair of arms 5A, 5B which are connected with each other by means of a hinge portion 7 so as to open/close and provided respectively with a blade 3 for spreading and holding an incision open at an end portion of the arms 5A, 5B, and a pair of handles 9A, 9B formed on the arms 5A, 5B for performing an open/close operation of the arms 5A, 5B, as in the case of the aforementioned conventional surgical retractors 1.

Also, the surgical retractors 1A are provided with bent arms 21A, 21B that bend downward in relation to the arms 5A, 5B at the end of the arms 5A, 5B, respectively, the arms 21A, 21B having at their own ends the blades 3. The tip or lower portion of each blade 3 bends away from each other (outward) at a position of the bend line L. Seen from a lateral side, the bend line L is formed so as to be substantially parallel with the plane P formed by the arm 5A, 5B and the handles 11A, 11A, as shown in FIG.3.

More particularly, the bent arms 21A, 21B bend downward at an angle of about 45 degree in relation to the arms 5A, 5B, as shown in FIG.3. At the end of the bent arms 21A, 21B is provided a blade back portion 3A that can be supported horizontally when supporting the arms 5A, 5B horizontally and extends from the arms 5A, 5B. In other words, it is understood from FIG.3 that the blade back portion 3A is formed so as to be parallel with the arm 5A, 5B, when the blade back portion 3A and the arms 5A are seen from their lateral side.

Moreover, the blade back portion 3A is provided integrally with a plural of finger portions 3B that are formed so as to extend downward and substantially orthogonal with the blade back portion 3A. The lower end of the finger portions 3B bends outward at the bent line L.

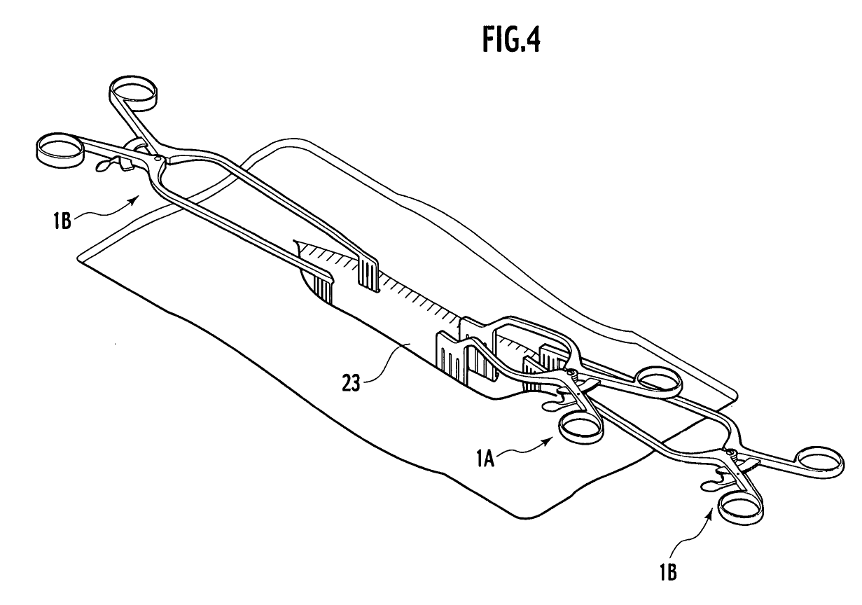

Such a configuration allows the following way of using the surgical retractors 1 when performing a surgical operation for a patient's spinal cord, for example (see FIG.4). When an incision 23 made on his or her back is spread widen, at first, the edge of the incision 23 is spread to a certain extent at a portion of the incision 23 adjacent to the end thereof using smaller sized surgical retractors 1B. Next, at the center portion of the incision 23, blades 3 of larger sized surgical retractors 1A are inserted deep into the incision 23 until the extremity portion of the blades 3 reaches beneath the skin. Then, the incision 23 is spread wide to an appropriate extent by closing the handles 9A, 9B and the arms 5A, 5B are locked with each other by the lock mechanism 13. In this situation, the arms 5A, 5B and handle 9A, 9B of the surgical retractors 1A can be overlaid partially on the smaller surgical retractors 1B. This can be realized because the bent arms 21A, 21B are bent downward from the arm 5A, 5B and also the bend line L at which the lower portions of the blades 3 are bent outward is substantially parallel with the arms 5A, 5B and handles 9A, 9B, seen from their lateral side.

In other words, even when a deep portion of the incision needs to be spread wide by inserting the blades 3 deep beneath the skin at the center portion of the incision 23, the arms 5A, 5B, the handles 9A, 9B, and the rings 11A, 11B are not raised upward higher but laid substantially horizontally, thereby preventing the arms 5A, 5B, the handles 9A, 9B and rings 11A, 11B from interfering with the surgical operation. This means that the aforementioned problem accompanied with a conventional type of surgical retractors is solved.

The surgical retractors according to the present invention are applicable in spreading and holding a surgical incision open.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. Surgical retractors provided with a pair of arms which are openable and closable via a hinge portion and include at an extremity portion thereof a blade for spreading and holding open an incision and a pair of handles for performing an open/close operation of said pair of arms, the pair of handles being provided on said pair of arms respectively, wherein there is provided at an extremity side of each of said arms a bent arm portion that bends downward in relation to the arm, and a bend line, formed at an extremity portion of said blade provided at an extremity end of the bent arm portion, is substantially parallel with said arm when seen from a lateral side.
